(19) United States
(12) Patent Application Publication
(10) Pub. No.: US 2002/0151774 A1
(43) Pub. Date: Oct. 17, 2002

(54) Ocular spectrometer and probe method for non-invasive spectral measurement

(75) Inventors: Babs R. Soller, Northboro, MA (US); Bilal Saleh, Worcester, MA (US); Edward Chaum, Germantown, TN (US); Markus E. Testorf, Lebanon, NH (US); Michael Fiddy, Chelmsford, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: UMASS/WORCESTER, Worcester, MA

(21) Appl. No.: 10/086,903

(22) Filed: Feb. 28, 2002

Related U.S. Application Data
(60) Provisional application No. 60/272,552, filed on Mar. 1, 2001.

(51) Int. Cl. A61B 5/00

(52) U.S. Cl. 600/318; 600/321

(57) ABSTRACT

A non-invasive spectral measurement of a native, diagnostic or treatment component in blood or tissue, illuminates the back of the eye and collects return light that has passed through and been reflected from choroidal or retinal tissue. Spectral analysis detects a retinal tissue state, or detects the level of a blood or serum constituent, which may be a native constituent or a dye, marker or pharmacological agent. Time-resolved or spectral decay monitoring may be used to assess organ functioning, e.g., by administering a serum-carried indicator of uptake, clearance or binding rate for specific organs. Circulating cells or material diagnostic of different conditions may also be detected by spectral analysis, either directly, or by tagging with a suitable label. A special probe which may include an ophthalmic lens is arranged to couple the return signal from the fundus into one or more collection fibers coupled to a spectrometer, and may be hand-held or mount directly on the front surface of the cornea, providing a simple clinical tool for non-invasive spectrometric access to the bloodstream, requiring little or no special training, and without resort to costly ophthalmic instrumentation.

### CROSS REFERENCE TO RELATED APPLICATIONS

This application claims priority from U.S. Provisional Patent Application Serial No. 60/272,552, filed on Mar. 1, 2001, entitled “Retinal Spectrometer and Probe Method for Non-Invasive Spectral Measurement,” which is expressly incorporated by reference herein.

### BACKGROUND OF THE INVENTION

Much interest has been expressed recently in spectroscopy, in particular infrared (IR) or near infrared red (NIR) spectroscopy, to non-invasively determine blood or tissue chemistry. Tissue in living subjects, e.g., in human patients, presents an extraordinarily complex medium, with many contributing absorbing and scattering materials present that affect the returned light signal. Factors such as temperature, or the drift of components or instrumentation, that may be successfully addressed in in vitro spectroscopy, may also result in variations of the sampled spectrum.

Some successes in correcting or interpreting spectra obtained from complex measurement environments have been reported by applying statistical methods. These statistical techniques and processing modalities, commonly referred to as chemometrics or multivariate calibration, reduce spectral variability to a linear combination of a small number of component spectra, which can then be used in a calibration equation for determining a clinical parameter of interest from an acquired spectrum. However, spectral results in vivo do not compare favorably to classical in vitro colorimetric measurements due to the presence of uncontrolled variability. Other techniques, directed more to detection than measurement, have been proposed that rely on correlation with large databases of non-specific tissue spectra.

Generally, for chemometric or multivariate calibration, the component spectra are derived empirically by simultaneously collecting a number of spectra together with reference measurements, taken at the time each spectrum is acquired, e.g., measuring a clinical parameter of interest as well one or more environmental parameters, such as temperature. In applying this technique, one hopes that the most significant spectral variability is due to the clinical parameter of interest, for example glucose concentration, and that the principal secondary effects have been correctly identified and either measured or modeled. However, when working in vivo, a great number of strongly absorbing or scattering influences, as well as other confounding influences may be present, resulting from a variety of structural and chemical constituents of the probed tissue environment and other contributing factors. Human variability also poses a large confounding influence on the shape and quality of the returned light signal. As a result, tissue spectrometry is presently of limited use, and the preponderance of spectrometric assays must still be effected by withdrawing and preparing blood or tissue specimens for ex vivo analysis, or for analysis by non-spectrographic techniques, e.g., in a blood analysis machine or a clinical kit. In vivo quantitative measurements, involving correction for specific factors, therefore tend to be achievable for a limited number of target components in certain well-defined environments, typically involving corrections for parameters such as temperature, instrument drift and other relatively objective factors.

In ophthalmology, it has also been known to visually or analytically assess certain analytes or indicators in vivo, when these are present in the blood stream, by directing light into the eye, at the retina or choroidal tissue, where thin vessels present direct optical access to flowing or capillary blood. This has generally been done in the context of ophthalmic treatment or diagnosis, using optics similar to those of a slit lamp or a retinal camera to project and collect light. For example, retinal vasculature is commonly assessed by flourescein angiography imaging, and measurements such as Doppler blood flow measurements have been performed by directly illuminating the fundus and collecting light reflected back therefrom. Such operations tend to be application-specific, and may require quite customized hardware to obtain suitable signals. By way of example, a probe may be required to focus to a spot size smaller than diameter of the retinal vessel, or to illuminate or collect along precisely-oriented optical paths to achieve meaningful Doppler data.

However, there remains no method of general applicability for making in vivo spectrometric determinations, and certain areas, such as assessment of internal organs or detection of circulating indicators of disease, have resisted efforts of clinicians to devise quick or non-invasive methods to carry out functional or diagnostic clinical evaluations.

Accordingly it would be desirable to develop an apparatus and a methodology for in vivo spectrometry, and in particular one that is diverse to different clinical parameters of interest. It would also be desirable to develop a spectrographic instrument and method that non-invasively assesses organ function, and that is effective for the accurate detection of a clinical parameter of interest through a subject's eye.

### SUMMARY OF THE INVENTION

One or more of these and other desirable features are attained in a method of the present invention for performing a non-invasive spectral measurement, and a probe adapted for practicing the method, wherein illumination and collection optics are applied to the eye of a subject and a spectrometer processes light collected from the eye to perform an assay or to evaluate a clinical state of interest.

The optics implement a non-invasive spectral measurement of a native, diagnostic or treatment component in blood or tissue, by illuminating the back of the eye and collecting return light that has passed through the vitreous and has interacted with and returned from retinal and choroidal tissue at the back of the eye. Spectral analysis is then applied to detect the level of a blood or serum constituent, that, in various embodiments, may be a native constituent or may be a dye, a marker or a pharmacological agent. It may also detect the spectral signature of a tissue condition present in the fundus.

In one embodiment, repeated spectral samples are taken to form a time-resolved spectral sequence indicative of the target component. Such time-resolved monitoring may be used to assess circulation, and/or may be used in conjunction with administration of an exogenous compound to assess organ function, e.g., by detection of a serum-carried indicator configured for uptake or clearance by, or binding with, a specific organ. A compound with suitable spectral properties may be first administered, e.g., having an organ-specific uptake, clearance or binding rate property, such that the temporal variation of the target spectral component represents organ function. The time-resolved spectral sequences (spectral decay) may also be used to assess cardiac or circulatory function.

In other embodiments, the invention assesses a circulating component, such as cells, or a protein or peptide produced by cells, to which a marker may have been applied or reacted with.

A probe adapted for the practice of the invention may include an optical assembly that mounts or is positioned in front of the eye and is arranged to illuminate the eye and couple return light to a collection fiber. The assembly may include an ophthalmic lens that mounts directly on the front surface of the cornea, e.g. with a gel layer, providing a simple clinical tool for non-invasive spectrometric assays of materials present in blood or serum. A positioning mechanism adjusts and/or moves the collection fiber to enhance light collection for different axial lengths and refractive errors. One embodiment may employ direct illumination and utilize a fiber only for collection, and other embodiments may be implemented as hand-held probes. In each case, the collection fiber couples to a spectrometer that processes the collected signal to develop spectral data for detection or quantification of a serum-carried component or material of interest.

### BRIEF DESCRIPTION OF THE DRAWINGS

These and other features of the invention will be understood from the description below and the claims appended hereto, taken together with drawings of illustrative embodiments, wherein

FIG. 1 is a perspective view illustrating one embodiment of a spectral probe according to the present invention;

FIG. 1A is a perspective view illustrating another embodiment of a spectral probe according to the present invention;

FIG. 2 is a flow chart illustrating the steps in a method of spectral analysis according to one embodiment of the present invention;

FIG. 3 is a graph illustrating a received ophthalmic spectra;

FIG. 4 is a flow chart illustrating a system for spectrographic analysis in accordance with the present invention;

FIG. 5 is a schematic diagram of a method for measuring the input light simultaneously with the light reflected from the eye.

### DETAILED DESCRIPTION

The present invention includes a spectral analysis system for non-invasive collection of spectrographic information indicative of serum components, and also includes novel methods for clinical assays using blood or serum spectrometry by optically accessing the vasculature of a patient along optical paths through the eye, e.g., with a spectral collection unit operated at the front surface of a patient's eye. Unlike conventional approaches to evaluating blood chemistry or systemic health traits of interest, the invention simply employs an optical probe configured to attach to or be held close to the cornea of a patient, and to couple a light signal from fundus tissue to a spectrometer. Unlike much ophthalmological instrumentation, the probe may simply couple or pass illumination, and collect a return signal from the retina and choroid, without necessarily imaging or even viewing the field of interest. The collected light is coupled from the probe to a spectrometer for direct spectrometric assessment of one or more serum components, or identification of constituents and/or concentrations of constituents that are present in the patient's blood stream. In other embodiments, the device includes a spectrometer attached to an ophthalmic spectral collection assembly, and configured to process collected light spectra or monitor spectral decay for assessment of serum carried components or indicators.

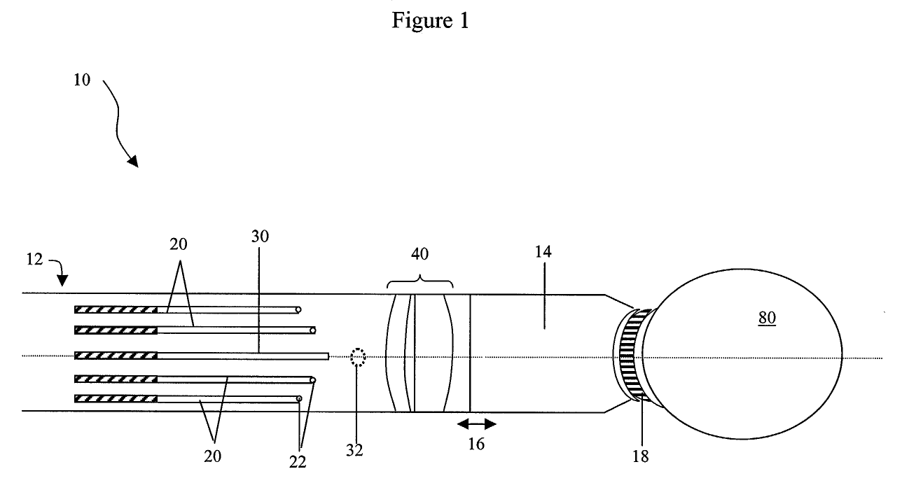

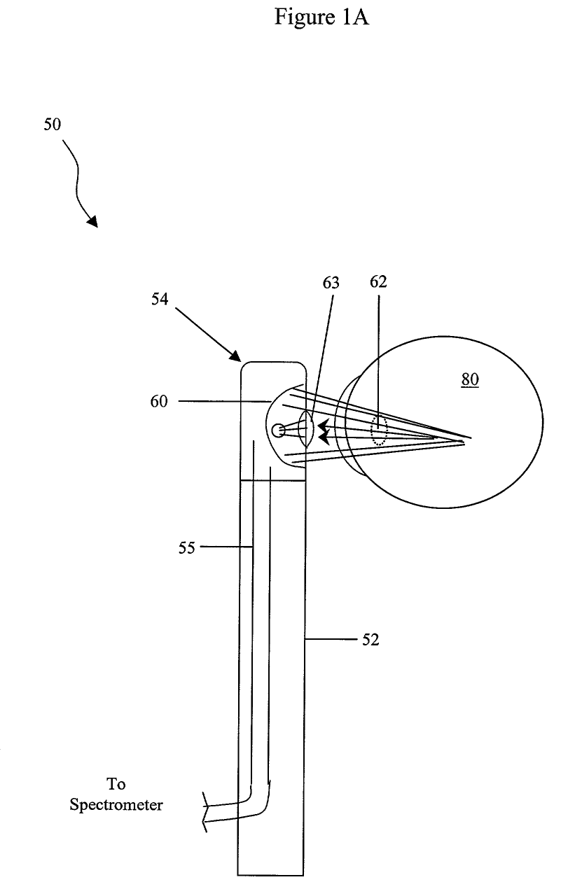

FIG. 1 illustrates one embodiment of a probe 10 intended for the practice of the invention. In general, one or more optical fibers are positioned at an optical assembly to deliver illumination to, and to collect return light from, the back of the patient's eye. As shown, the probe 10 includes a body 12 having a plurality of fibers 20, 30 disposed therein, a collimating lens 40, a coupling assembly 16, and an optical assembly 14.

The fibers 20, 30 preferably include a collection fiber 30, which can be formed from a plurality of optical fibers, and one or more illuminating fibers 20, or similar type of light source. The illuminating fibers 20 are preferably positioned symmetrically around the collection fiber 30, and are independently movable with respect to the collection fiber 30 to allow for greater flexibility. The probe 12 can also optionally include one or more calibration rods to support the fibers 20, 30 and to provide a means for calibration to measure the distance of the collecting fiber 30 and the illumination fibers 20 from the collimating lens 40.

The positioning/coupling assembly (indicated schematically by arrow 16) is effective to position the fibers 20 with their end face(s) arranged in a collection region 32 centered at a focus in the probe body 12. The optical assembly 14 is disposed at the distal most end of the body 12 and includes an ophthalmic lens (not shown) for positioning the probe 10 on the cornea. The collimating lens 40 is positioned between the fibers 20, 30 and the coupling assembly 16, and is effective to focus a reflected image of the back of the eye onto the collecting fiber 30.

In use, the probe 10 is placed on the eye and the illuminating fiber ends 20 are positioned to transmit light into the choroidal or fundus tissue at the back of the eye. Preferably, the ophthalmic lens is positioned 5 mm from the surface of the cornea. The ends of the light-receiving fibers 30 in the detection area may be separated from the illumination fibers 20 by a distance corresponding to a few millimeters (as projected on the retina) to assure an effective level of spectral interaction with tissue at the back of the eye for forming an effective probe. The ophtalmic lens collects the reflected image, and the collimator lens 40 is then used to focus the image onto the collecting fiber 30. In this embodiment, illumination is provided by a fiber or plurality of optical fibers 20 with their light-emitting ends located in a region 22 that is spaced extending around the center of the probe head. A gel layer 18, such as an index matching gel, can be provided for keeping the eye moist and coupling the face of the ophthalmic lens on the optical assembly 14 to the cornea, and to establish index matching between the imaging lens and the eye 80.

FIG. 1A illustrates another embodiment 50 of a probe suitable for use with the present invention. As shown, probe 50 includes a hand-held body 52 having an optical probe head 54 that illuminates and collects return light from the retina. A collection fiber assembly 55 having an illumination source is provided and is effective to mate to a spectrometer. As shown, illumination can be directed from the optical assembly 55 into the pupil of an eye 80 from a speculum-like projection surface 60 having a generally annular region. The light reflected back from the retina is received along a generally central path 62 into a collimating lens 63, whereby the light is directed to the collection fiber in the collection fiber assembly 55 to be transmitted to the spectrometric instrumentation. The illustrated device 50 arranges the illumination and collection paths to avoid direct reflection of the illuminating light into the collector from intervening curved reflectors (e.g., the anterior corneal surface, and surfaces of the natural lens), while assuring that the collected signal emanates from the tissue illuminated at the back of the eye.

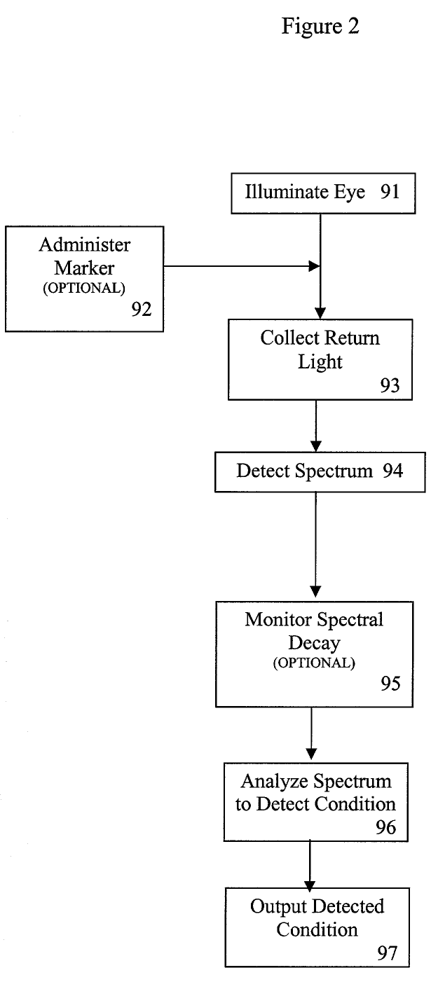

Thus, the probes according to the present invention are devices that permit illumination of the vascularized tissue of the retina and choroid of the eye, and that collect and analyze the light which is reflected or returned back. FIG. 2 is a flow chart illustrating the general steps for using the devices according to the present invention. As shown, the device is placed on the eye and the eye is illuminated 91. A marker can optionally be administered 92. The return light is then collected 93 and the spectrum is detected 94. Spectral decay can optionally be monitored 95 at this point. The spectrum or collection of spectra is analyzed to calculate the parameter or parameters of interest 96. The processing unit connected to the spectrometer then outputs the detected condition 97 for evaluation.

In some embodiments, the collection assembly can be directly coupled to a spectrometer. For example, the collection assembly can direct the collected light to a slit of a spectrometer having a wavelength dispersing element to generate a spectrum.

The light return signal may be a result of light which was not absorbed by blood or dyes in the blood (reflectance spectrum), or alternatively may be from a fluorescing or phosphorescing dye or marker material that is circulating in the patient's blood stream. Such markers or indicator materials are used to effect direct spectrometric assessment of serum assays, to indicate status of a tissue site or a remote organ or cellular components, proteins or peptides circulating in the blood, or to directly assess a retinal or choroidal tissue state or an entirely non-ophthalmic health condition.

Methods of the invention may employ an ophthalmic collection assembly to provide medical assessment in a number of ways. Through the reflectance spectrum of the blood it is possible to measure a condition or the concentration of a constituent such as arterial blood pH, blood gases (partial pressure of oxygen and carbon dioxide), blood bicarbonate or lactate concentrations, hemoglobin oxygen saturation, glucose, sodium, potassium, calcium, and hemoglobin/hematocrit. By way of example, reference is made to U.S. Pat. Nos. 5,813,403 and 6,006,119 relating to optical measurement of tissue pH and hematocrit. Through the analysis of the optical properties of circulating dyes it is also possible to non-invasively study internal organ/cell function. This may be done, for example, using a dye that is selectively taken up by an internal organ. Several such dyes are currently FDA approved for medical use, and can be utilized in practices of the invention to assess internal physiology.

For instance, in accordance with one practice of the present invention, liver function is assessed by measuring absorbance of the dye indocyanine green (ICG) at 804 nm. To perform this measurement, ICG is injected into the blood stream, so that over time it is cleared by the liver. The retinal monitor detects decay of the ICG spectrum, or selected wavelengths of the spectrum over time. The rate of decline of ICG absorption in the blood of the eye is related to both cardiac output and to the rate of clearance of ICG by the liver. Thus, when there is either impaired circulation, or liver dysfunction, ICG is cleared more slowly.

Systems of the invention may operate using a database of ICG clearance curves compiled for different levels of cardiac function and liver function, and/or may operate by calculating a rate of change of absorbance at one or more specific wavelengths (e.g., 804 nm) over a time period after a dye has been administered. A system may include a processor that compares the monitored spectra to arrive at assessments of cardiac and/or liver function. Similarly, other medical dyes and molecular markers can be used (when available), or may be designed, to probe other specific health conditions, or cellular or organ functions. Sodium fluorescein dye fluoresces at 525 nm, and this compound is cleared rapidly and almost exclusively by the kidney. Thus, when first administered, the rate of decline in retinal fluorescence is an indirect function of renal clearance of the dye, and can be detected by the probe 10 and applied to measure kidney function.

In a similar manner, the concentration and clearance of any drug or blood chemical or protein or peptide which has a definable absorption and/or emission spectrum or can be tagged with a luminescent material can be measured by monitoring the amplitude of its retinal and choroidal reflectance, absorption, or emission spectrum using this method of retinal spectrometry, or by measuring spectral decay of the target. For instance, when a disease state produces characteristic circulating cells, proteins, or peptides, those cells or materials can be specifically tagged with absorbing or emitting dyes or other luminescent materials, for example, using known or readily determined antigen binding material or labeling technology, so that the spectral signature of the dye in the retinal probe signal allows their detection through the eye. The materials used to selectively label blood circulating elements may be introduced into the bloodstream through any of a number of methods including, but not limited to, injection directly into the blood stream, oral administration in the form of a pill or liquid, transdermally via a chemical releasing patch, or nasally via administration of an aerosolized form of the marking agent.

The device may also have applications in the direct measurement of conditions such as retinal pH and oximetry. Retinal pH and ischemia are important clinical parameters in the development of retinopathy in conditions such as diabetic macular edema, proliferative retinopathy and retinal vascular occlusive disease. The ability to monitor local tissue parameters in the retina is of potential value in the management of diabetic retinopathy, and provides a useful extension of the currently available clinical indicia of retinal diseases. The presence of edema may be recognized in the collected signal by distinctive changes in shape of the spectrum due to decreased level of tissue scattering and water absorption, while the spectral changes correlated with pH may be identified in a number of ways as described in the aforesaid patents of B. Soller et al.

Other methods for use with the present invention are disclosed in a U.S. Patent application entitled “Correction of Spectra for Subject Diversity,” by Babs R. Soller and Patrick Idwasi, filed on Feb. 28, 2002. This application, which is hereby incorporated by reference, discloses a non-invasive spectral measurement for a target analyte present in a subject's tissue or blood which derives spectral shapes corresponding to one or more human variability factors, such as skin color, from spectra collected from a diverse calibration group of subjects. Another set of spectra are normalized based on the derived spectral shapes to generate a set of corrected spectra. The corrected spectra are then utilized to generate and/or enhance a calibration model for detecting and/or measuring the target analyte from one or more subject spectra that are obscured by a human factor such as melanin, which is present in the retinal pigmented epithelium of the eye.

The present invention also provides a number of useful construction details and structural variations for a prototype probe device. In general, in one initial embodiment, light illuminates the retina and choroid through four fibers. Return light from the retina and choroid is shaped by the opthalmic lens and with a collimating lens assembly focused into a detecting fiber which is coupled to an optical spectrometer. The spectrometer performs spectral analysis of the collected light from the back of the eye. Output from the spectrometer is directed to a microprocessor which processes the spectrum according to a predetermined algorithm or algorithms to detect the disease condition or clinical analyte(s) of interest.

By way of non-limiting example, the device can include illumination fibers having a 300 μm core diameter for increased light delivery. One example of a suitable fiber is an APC 300/400N Anhydroguide PCS Nylon Fiber manufactured by Fiberguide® industries. In an alternative embodiment, the illumination fibers can be replaced by one or more light sources, such as a miniature light bulb, an ophthalmic examination light source, or a Luxtec surgical light source. One example of an exemplary light source is an LS-1 Tungsten Halogen light source manufactures by Ocean Optics®. The LS-1 light source is a white-light source optimized for use at 360 nm-2 mm. The lamp offers high color temperature and has a sufficient output and life span. The lamp also includes an SMA 905 connector for easy coupling. In another embodiment, a miniature light source having a suitable bandwidth is mounted between the ophthalmic lens and the patient's cornea. This configuration would ensure the passage of light directly to the retina and eliminate potential positioning instability.

The collection fiber 30 is preferably a plastic clad silica fiber suitable for high transmission efficiency. In an exemplary embodiment, the fiber is a tapered fiber having different core diameters at both ends. Preferably, the diameter is greater at the detection end and smaller at the end that is coupled with the spectrometer. One example of a suitable collection fiber is an APC 100/200N Anhydroguide PCS Nylon Fiber manufacture by Fiberguide® Industries. The fiber preferably has a numerical aperture of about 0.40 for efficient light collection from extended sources, and a 100 μm core as may be required by the spectrometer's channel size. The mechanical strength (using the bend method) is preferably between about 50 and 70 Kpsi, and the recommended minimum bend radius is about 100 times the fibers diameter (momentary), and 200 times the fiber diameter (long term). Another example of a suitable collection fiber for use with the present invention is a 0.39-NA TECS™ Hard Clad Multimode Fiber (600/630/10401 μm) also manufactured by Fiberguide® industries. The fiber has a low OH for a visible to near-IR transmission, and a high numerical aperture for an efficient light coupling and superior transmission in tight bends.

A variety of suitable ophthalmic lens can be utilized with the present invention. The lens should be effective for indirect imaging, which is required to collect light reflected back from the retina and choroid as a form of an image that can be further collimated into an optical fiber. In one embodiment, the ophthalmic lens provides ultra wide field viewing and small pupil ability. Preferably, the lens has a mobile flexibility of about +/−3 mm of horizontal separation from the cornea, and has a light transmission percentage of about 99% or higher. One example of a suitable lens is a Super VitreoFundus™ lens manufactured by Volk®. The lens is made of HI/LD glass to ensure high resolution images, and delivers a 124° dynamic filed of view with 0.57×magnification. The lens is designed to scan the eye and collect the retinal reflected light exiting from the pupil in the form of a circular image. In an exemplary embodiment, the lens has a diameter of about 25 mm, and is positioned at a distance of about 5 mm. Other suitable lenses include the 90D Classic or the SuperField NC®, both manufactured by Volk.

A person having ordinary skill in the art will appreciate that a variety of suitable collimating lens can be used with the devices according to the present invention. One example of a suitable collimating lens for use with the present invention is a TechSpec™ manufactured by Edmund Industrial Optics. Such a lens includes a positive low index element and a negative high index element secured together to form an achromatic doublet which is computer optimized to correct for on-axis spherical and chromatic aberrations. The lens has a diameter of 25 mm, an effective focal length of 35 mm, and a back focal length of about 28 mm. The lens is mounted in a sliding tube that moves horizontally with respect to the ophthalmic lens with a calibrated distance to identify the optimal separation between both lenses. Another example of a suitable collimating lens is a Plano-Convex Lens by Edmund Industrial Optics. The Plano-Convex Lens has a positive focal length and coated versions have optimum light throughput.

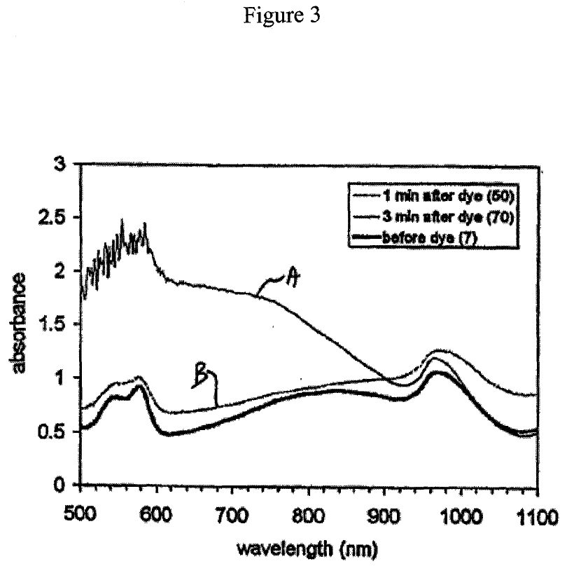

FIG. 3 illustrates preliminary spectral results from a device configured in accordance with the present invention and used on a pig eye. The heavy line shows the absorption spectrum of retinal and choroidal blood. The doublet centered at 540 nm is a good indication that a major component in the collected signal is oxyhemoglobin, which presents with good resolution (hence spectrometrically measurable signal) in the visible portion of the spectrum. The peak at 820 nm is also due to oxyhemoglobin. As is also shown in FIG. 3, the dye indocyanine green (ICG) was injected into the pig's vein and after one minute (as shown by the medium line “A”) one sees spectral evidence of the dye in the blood. ICG has an absorbance maximum centered around 800 nm. As the liver clears the dye from the blood (about three minutes after injection), the absorbance of the dye decreases (light line “B”).

The spectral system to which the probe attaches may operate in one of several ways. A broadband tungsten light source may be used to feed the illuminating fibers, or may be aimed directly into the eye, while the light reflected from underlying tissue (with wavelengths in the ultraviolet, visible or NIR band) is collected and directed to a spectral detector of a spectrometer. The spectrometer may be a scanning spectrometer, or may have a dispersion element such as a grating that both separates and directs a single return beam to a photo detector, such as a CCD array, to resolve and provide output values for the wavelengths of the spectral band. Alternatively, the spectrometer may illuminate with a broad band beam, and spectrally decompose the collected light analytically for example, by Fourier transformation techniques. Yet another construction is to employ a dispersive element to separate different wavelengths, scan a wavelength-varying component into the illumination fibers 20, and then simply employ a single detector (rather than a CCD or array) to measure the amplitude of the collected signal as a time-varying function of the scanned input illumination.

In some embodiments, the probe may include a detector placed at the probe collection region, rather than positioned at the distal end of a collection fiber assembly 30 leading from that region. However, fiber collection is preferred to allow the photoelectric detector element to be placed remotely in a well controlled environment, so that it may operate as a low-noise cooled assembly to achieve high signal to noise levels and high resolution. Typically the collection fiber couples directly to an existing spectrometer, and preferably the probe itself is a simple contact or optical projection/collection assembly implemented as a small hand-held probe, with all active spectral processing carried out by a separate console-type detector/processor unit. In one embodiment, the device may employ direct illumination, rather than relying upon a fiber assembly 20 for light delivery to illuminate tissue at the back of the eye. In this case, a beam may be directed, e.g., through a central or annular mask through the optical assembly (e.g., lens 14) to the eye, so the probe optics maintain a fixed geometry between the collector fiber and probed choroidal tissue region.

In other embodiments, the system may be optimized to detect luminescence (fluorescence or phosphorescene) from markers administered to detect specific cells, proteins and/or peptides in the blood circulation. By way of non-limiting example, the illumination may be provided by a narrow bandwidth light source, such as a light emitting diode, laser diode , pulsed laser, or filtered lamp, with emission intensity centered on or near the excitation maximum of the administered marker agent. The light emitting output of the marking agent may be detected by a single detector appropriately synchronized to detect light emitted from the marking agent and not from the incident light source. Alternatively, excitation may be provided by the illumination system described above and detection can be achieved through standard spectroscopic instruments and methods, such as those described above, applied with appropriate timing and synchronization as known to those having ordinary skill in this field.

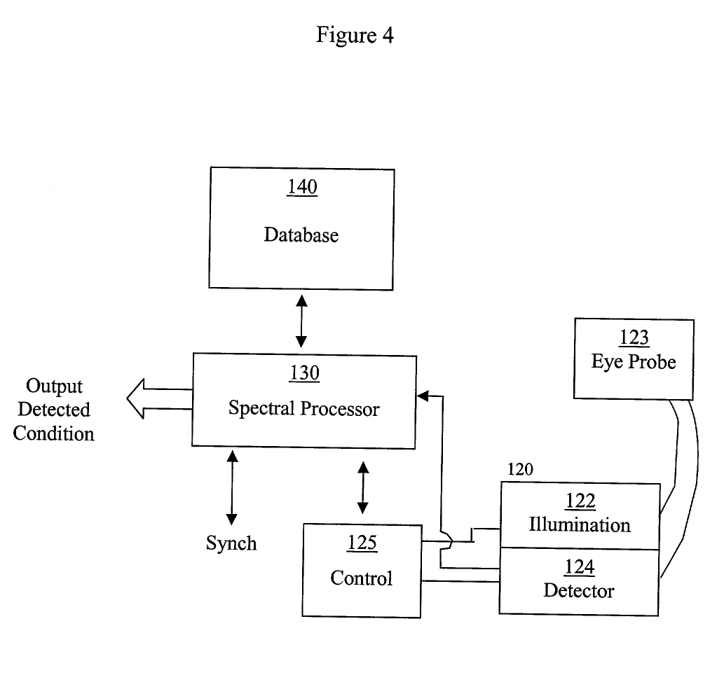

The elements of a spectrometer system for use with the present invention are shown in FIG. 4. As shown, an eye probe assembly 123 is connected to a spectrometer 120 having an illumination component 122 and a detection component 124 coordinated by a control unit 125. The control unit 125 may perform timing, scanning or normalizing operations appropriate for the type of spectrometer employed. The apparatus also includes a microprocessor-based spectral processor 130 operative on the detector 124 output, that processes the received spectral output, possibly applying various stored or look-up operations or multivariate analysis to correct for spectral components present in the retinal environment and to provide an enhanced assay of the dye or other target component. The processor 130 may communicate with one or more databases 140 that represent or model various spectral targets and diagnostic regimens and interpretations. It may also implement various processing or recognition routines (e.g., spectral analysis, fitting or matching operations or the like) to detect the material or conditions of interest. The processor may also generate or interface with suitable extrinsic controls or devices described above for marker injection and timer-resolved sampling, for example to effect dye injection, to synchronize signal gathering or processing with the injection or with cardiac or pulsatile signal detection, and other steps discussed above.

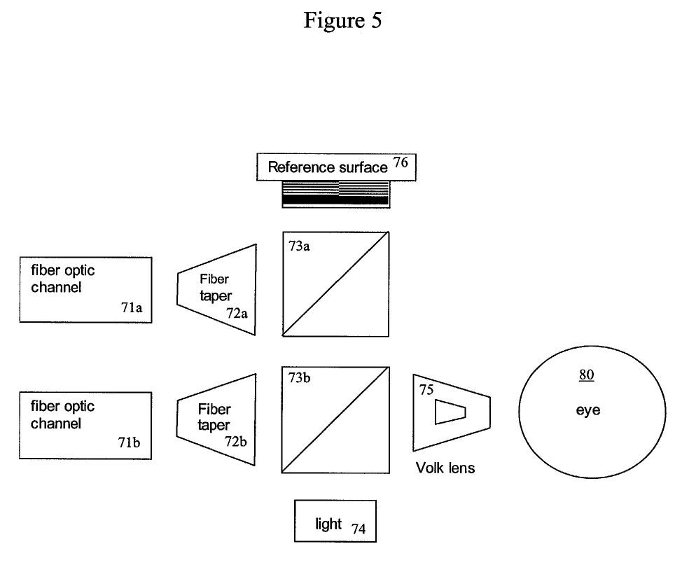

In a further embodiment of the present invention, a method is provided for measuring the input light simultaneously with the light reflected from the eye to achieve a stabilized and accurate signal by accounting for any light source fluctuations. As shown in FIG. 5, the probe can employ two beam splitters 73a, 73b. One beam splitter 73b directs a portion of incident light from the light source 74 to the eye 80, and allows another portion to be directed to the beam splitter 73a. The light backscattered or reflected from the eye 80 is returned through the Volk lens 75 to the fiber taper 72b to be transmitted through the fiber optical channel 71b to the spectrometer (not shown). The second beam splitter 73a allows the incident light from the light source 74 to propagate to a reference surface 76. Further, the beam splitter 73a directs the light reflected from the reference surface 76 to a second input in the spectrometer via fiber taper 72a and fiber optical channel 71a. The intensity of the light reflected from the reference surface, a reflectance standard, such as Spectralon, provided by Labsphere, Inc., provides a signal that allows monitoring the stability of the light source 74 in real-time, for example, throughout a measurement period. This reference signal can be utilized to normalize the absorption measurements for fluctuations in the light source, thereby reducing unwanted signal variation which may occur from light source instability. In another embodiment, the beam splitter 73a may be replaced by a chopper or rotating reflector which alternately directs light to the eye and the reference reflectance surface. Light detection by the spectrophotometer can be synchronized with the frequency of the light directing element. In this implementation the second channel of beam splitters 73a, fiber taper 72a and fiber optic channel 71a can be eliminated.

The devices of the present invention can also include a method for providing precise alignment of the probe with the cornea of the eye. This is preferably achieved by using an active feedback loop to position the instrument. The area under the spectral curve for each reading taken at a different position of the probe on the cornea can be calculated. This data can be then be employed to correlate an optical placement of the probe to a particular integrated area of the spectral response curve obtained with the desired or optimal placement. This correlation database can then utilized in a feedback loop to optimally position the probe on a subject's cornea. In particular, upon placement of the probe, the integrated area associated with the spectral curve is calculated in real time and is compared with the values in the correlation database. If the comparison shows a deviation from a desired value, the probe is moved until the desired value is obtained.

As noted above the probe of the invention need not connect to the eye, and it may be embodied as a hand-held unit, that connects, via optical fiber, to the spectral analysis instrumentation. However, illumination need not be provided by fiber delivery, and small light sources may be substituted, or direct spectral illumination from a large area source may be used. The construction illustrated in FIG. 1 has the advantage that illumination and collection fibers are imaged to closely adjacent regions of tissue, enhancing the spectral signal of interest, and that the optical paths are substantially separate, reducing the amount of illumination glare returned to the collection fiber 30. Further, the size of the collection fiber or fiber assembly may be increased to ensure collection of an adequate signal for spectrometric use.

The invention being thus described, variations and modifications will occur to those skilled in the art, and all such variations and modifications are considered to be within the scope of the invention, as described herein and encompassed within the claims appended hereto and equivalents thereof.

What we claim is:

1. A probe for non-invasive spectral assay of a serum-carried component, comprising:
a body having a proximal end, a distal end, and an inner lumen extending therebetween;
a light collection assembly disposed within the inner lumen of the body and being adapted to communicate with an spectrometer for spectral analysis of collected light;
an illumination assembly mated to the body and being effective to provide light; and
an optical assembly mated to the distal end of the body and positionable at the cornea of a subject's eye, the optical assembly being configured to direct light from the illumination assembly through a subject's eye to tissue and to direct return light from the tissue into the collection assembly.

2. The probe of claim 1, further comprising a processing unit in communication with the collection assembly for analyzing the return light.

3. The probe of claim 1, wherein at least one of the illumination assembly and the light collection assembly is a fiber optic assembly.

4. The probe of claim 1, wherein the light collection assembly comprises at least one collection fiber.

5. The probe of claim 4, wherein the illumination assembly comprises a plurality of illumination fibers.

6. The probe of claim 5, wherein the at least one collection fiber and the plurality of illumination fibers are disposed within the inner lumen of the body, and wherein the plurality of illumination fibers are positioned symmetrically around the at least one collection fiber.

7. The probe of claim 1, further comprising a coupling assembly for slidably moving the collection assembly with respect to optical assembly.

8. The probe of claim 1, wherein the optical assembly includes an ophthalmic lens effective to position the probe on a subject's eye and to form an image of the return light.

9. The probe of claim 8, further comprising a collimating lens disposed between the collection assembly and the optical assembly, the collimating lens being effective to focus reflected light from a subject's eye to the collection assembly.

10. The probe of claim 1, wherein the tissue comprises choroidal or fundus tissue.

11. The probe of claim 1, wherein the illumination assembly is selected from the group consisting of at least one optical fiber, a miniature light bulb, a surgical light source, and a ophthalmic examination light source.

12. A probe for non-invasive spectral assay of a serum-carried component, comprising:
a body;
a light collection assembly disposed within said body, said light collection assembly configured to couple to a spectrometer; and
an optical assembly mated to said body for coupling a return light signal from choroidal or fundus tissue of a subject's eye into the light collection assembly, such that the return light signal includes a spectrum of a target component carried in blood or serum;
whereby the spectrometer processes the light signal to directly assay said serum-carried component.

13. The probe of claim 12, wherein the collection assembly comprises an optical collecting fiber to couple to a spectrometer.

14. The probe of claim 12, wherein the optical assembly includes an ophthalmic lens effective to position the probe on a subject's eye.

15. The probe of claim 12, further comprising an illumination assembly for directing light into a subject's eye.

16. The probe of claim 15, wherein the body further includes a speculum-shaped projection surface for directing light from the illumination assembly into a subject's eye.

17. The probe of claim 12, wherein the optical assembly includes a collimating lens for directing reflected light to the optical collecting fiber.

18. A method of performing a non-invasive measurement of a target component present in blood or tissue comprising the steps of:
positioning an optical structure at the front of a subject's eye;
directing illumination into the subject's eye at fundus tissue;
collecting a light signal returned from said tissue; and
processing a spectrum present in the light signal to measure a targeted serum-carried component present in said tissue.

19. The method of claim 18, wherein said spectrum includes light selected from the group consisting of visible light, ultraviolet (UV), near infrared (NIR), and combinations thereof.

20. The method of claim 18, wherein said structure includes an opthalmic lens and at least one optical fiber, and wherein at least one of the steps of directing and collecting includes coupling light between the fibers and the back of the subject's eye.

21. The method of claim 18, wherein the step of processing includes time-resolved processing.

22. The method of claim 21, further comprising the step of administering to the subject an indicator of organ function, and wherein the step of time-resolved processing tracks serum concentration of the indicator thereby assessing organ function.

23. The method of claim 22, wherein the indicator is an indicator taken up by or cleared by an organ.

24. The method of claim 22, wherein the indicator is an indicator selectively taken up by or depleted by a liver or kidney.

25. The method of claim 22, wherein the indicator is an indicator selectively taken up by or depleted by an organism or diseased tissue.

26. The method of claim 18, wherein the step of processing includes correcting the spectrum for a contributing factor.

27. The method of claim 22, wherein the step of time-resolved processing tracks serum concentration of an indicator to assess cardiac function or circulation.

28. The method of claim 27, wherein the step of time-resolved processing tracks serum concentration of a tagged or untagged therapeutic agent.

29. The method of claim 18, wherein said structure is arranged to position illumination and collection light along substantially non-interfering paths through the subject's eye so as to reduce scattering noise in the collected light.

30. The method of claim 18, wherein the step of processing includes monitoring a spectral signal of any of a tagged cellular or serum component, a marking agent administered invasively, and a marking agent administered non-invasively.

31. The method of claim 18, wherein the step of directing illumination into the subject's eye further comprises the simultaneous step of directing illumination to a reference surface to account for light source fluctuations.

32. The method of claim 18, wherein the step of positioning the optical structure on the front of the subject's eye is repeated after the steps of collecting and processing to achieve optimal alignment of the optical structure with the subject's eye.

33. An optical system for non-invasive spectral assay of a serum-carried component, comprising:
a source for providing light;
first and second beam splitters optically coupled to the light source, the first beam splitter directing a portion of the light to a subject's eye to elicit reflected light from the eye, and directing a second portion of the light to the second beam splitter;
a reference reflection surface optically coupled to the second beam splitter to be illuminated by the second light portion, said surface directing reflected light in response to said illumination to the second beam splitter; and
first and second collection assemblies coupled to said first and second beam splitters, respectively;
wherein the first beam splitter directs the light reflected from the eye to the first collection assembly, and the second beam splitter directs the light reflected from the reflection surface to said second collection assembly.

34. The optical system of claim 33, further comprising a processor coupled to said first and second collection assemblies, the processor normalizing the reflected light from the subject's eye relative to the light reflected from the reference surface to correct for fluctuations in the light emitted by the light source.

35. The optical system of claim 34, wherein the processor normalizes the light reflected from the subject's eye in real time.

36. A method of performing a non-invasive measurement of a target component present in the blood or tissue, such as a native, diagnostic or treatment component, wherein the method comprises the steps of:
a) positioning an optical assembly at a front surface of a subject's eye to collect a light signal from choroidal tissue at the back of the eye;
b) coupling the collected signal to a spectral processor; and
c) processing a spectrum present in the collected light to directly measure a targeted serum-carried component or a condition present in said tissue.

37. The method of claim 36, wherein the collected light signal is processed to detect a spectrum of a serum-carried component indicative of a health condition or disease state.

38. The method of claim 37, wherein said health condition or disease state is any of non-ophthalmic and ophthalmic disease.

39. The method of claim 37, wherein said targeted serum-carried component is an indicator of disease state.

40. The method of claim 37, wherein said target serum-carried component includes at least one of circulating cells, a marker material, proteins, and peptides.

41. The method of claim 37, wherein the step of processing a spectrum includes monitoring dye kinetics.

42. The method of claim 37, wherein the collected signal includes a visible and/or UV and/or NIR component.

43. The method of claim 37, wherein the collected light includes at least one of a reflectance, a fluorescence and a phosphorescence component.

44. An optical system for non-invasive spectral assay of a serum-carried component, comprising:
a source for providing light;
an optical member coupled to the source for alternatively switching the light propagation direction between a first direction and a second direction, the first direction illuminating a subject's eye;
a reference reflectance surface optically coupled to the optical member to be illuminated by light propagating in the second direction, the surface providing reflected light in response to said illumination;
a detector that detects light reflected from the subject's eye and the reflectance surface.

45. The optical system of claim 44, wherein the optical member can be any of a chopper, rotating mirror, and a beam-splitter.
